(19) United States
(12) Patent Application Publication
(10) Pub. No.: US 2009/0291961 A1
(43) Pub. Date: Nov. 26, 2009

(54) COMBINATION OF AN MEK INHIBITOR AND THE SRC KINASE INHIBITOR AZD0530 FOR USE IN THE TREATMENT OF CANCER

(75) Inventors: Neil Oliver Carragher, Leicestershire (GB); Tim Paul Green, Macclesfield (GB); Paul David Smith, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/520,639

(22) PCT Filed: Dec. 20, 2007
(86) PCT No.: PCT/GB07/04927

§ 371 Date: Jun. 22, 2009

(30) Foreign Application Priority Data
Dec. 22, 2006 (GB) 0625691.1

(51) Int. Cl. A61K 31/517; A61P 35/00

(52) U.S. Cl. 514/252.17

(57) ABSTRACT

The invention relates to a combination for use in the treatment of cancer comprising a MEK inhibitor and the Src kinase inhibitor AZD0530.

In a first aspect, the present invention relates to a combination comprising a MEK inhibitor and a particular inhibitor of the Src family of non-receptor tyrosine kinases. The combination of the invention is useful in a method for the treatment of cancer. The invention also relates to a pharmaceutical composition comprising such a combination and to the use thereof in the manufacture of a medicament for use in the treatment of cancer or in the manufacture of a medicament for use in the delay of the progression of cancer.

Current options for treating cancer include surgical resection, external beam radiation therapy and/or systemic chemotherapy. These are partially successful in some forms of cancer but are less successful in others. There is a clear need for new therapeutic treatments for treating cancer.

In recent years it has been discovered that a cell may become cancerous by virtue of the transformation of a portion of its DNA into an oncogene i.e. a gene which, on activation, leads to the formation of malignant tumour cells (Bradshaw, Mutagenesis, 1986, 1, 91). One of the key attributes of malignant cells is the ability to migrate and invade and migrate surrounding tissues leading to host tissue destruction and the formation of secondary metastatic lesions. To achieve this tumour cells must acquire a motile and invasive phenotype as a result of the oncogenic activation of a variety of signalling pathway components. Oncogenes give rise to the production of peptides which are receptors for growth factors. Activation of the growth factor receptor complex subsequently leads to an increase in cell proliferation, motility and invasion. Oncogenes often encode abnormal versions of signal pathway components, such as receptor tyrosine kinases, serine-threonine kinases, or downstream signaling molecules such as the ras genes. The ras genes code for closely related small guanine nucleotide binding proteins which hydrolyse bound guanosine triphosphate (GTP) to guanosine diphosphate (GDP). Ras proteins are active in promoting cell growth, transformation and invasion when they are bound to GTP and inactive when they are bound to GDP. Transforming mutants of p21 ras are defective in their GTPase activity and hence remain in the active GTP bound state. The ras oncogene is known to play an integral role in certain cancers and has been found to contribute to the formation of over 20% of all cases of human cancer.

When activated by ligand, such as a growth factor, cell surface receptors which are coupled to the mitogenic response can initiate a chain of reactions which leads to the activation of guanine nucleotide exchange activity on ras proteins. When ras protein is in its active GTP-bound state, a number of other proteins interact directly with ras at the plasma membrane resulting in signal transmission through several distinct pathways. The best characterised effector protein is the product of the raf proto-oncogene. The interaction of raf and ras is a key regulatory step in the control of cell proliferation. Ras-mediated activation of the raf serine-threonine kinase in turn activates the dual-specificity MEK (MEK1 and MEK2), which is the immediate upstream activator of mitogen activated protein kinase (MAPKs known as extracellular signal regulated protein kinases or ERK1 and ERK2). To date, no substrates of MEK other than MAPK have been identified, though recent reports indicate that MEK may also be activated by other upstream signal proteins such as MEKK1 and Cot/Tpl-2. Activated MAPK translocates and accumulates in the nucleus, where it can phosphorylate and activate transcription factors such as Elk-1 and Sap1a, leading to the enhanced expression of genes such as c-fos. In addition, activated MAPK also phosphorylates other kinases eg p90RSK and cytoskeletal proteins.

The ras-dependent raf-MEK-MAPK cascade is one of the key signalling pathways responsible for conveying both mitogenic and invasive signals from cell surface to the nucleus resulting in changes in gene expression and cell fate. Transforming mutants of p21ras are constitutively active, resulting in raf, MEK and MAPK activity and cell transformation. Inhibition of MEK activity using either antisense raf, a dominant negative MEK mutant or the selective inhibitor PD098059 has been shown to block the growth and morphological transformation of ras-transformed fibroblasts, cell motility and invasion.

The mechanism of activation of raf, MEK and MAPK is through phosphorylation on specific serine, threonine or tyrosine residues. Activated raf and other kinases phosphorylate MEK1 on S218 and S222 and MEK2 on S222 and S226. This results in MEK activation and subsequent phosphorylation and activation of ERK1 on T190 and Y192 and ERK2 on T183 and Y185 by the dual specificity MEKs. Whilst MEK can be activated by a number of protein kinases, and active MAPKs phosphorylate and activate a number of substrate proteins including transcription factors, other protein kinases and cytosolic proteins, some of which are implicated in the invasive process, MEKs appear specific and sole activators of MAPKs and could act as a focal point for cross-cascade regulation. MEK1 and MEK2 isoforms show unusual specificity and also contain a proline-rich insert between catalytic subdomains 1× and X which is not present in any of the other known MEK family members. These differences between MEK and other protein kinases, together with the known role of MEK (MEK 1, MEK 2) and, more recently MEK 5, in proliferative and invasive signalling suggest it may be possible to discover and employ selective MEK inhibitors as therapeutic agents for use in proliferative and invasive disease.

Accordingly, it has been recognised that an inhibitor of the MAPK kinase pathway should be of value both as an anti-proliferative and anti-invasive agent for use in the containment and/or treatment of solid tumour disease.

It is also known, for example, that several oncogenes encode tyrosine kinase enzymes and that certain growth factor receptors are also tyrosine kinase enzymes. The first group of tyrosine kinases to be identified arose from such viral oncogenes, for example pp 60^{v-Src} tyrosine kinase (otherwise known as v-Src) and the corresponding tyrosine kinases in normal cells, for example pp 60^{C-Src} tyrosine kinase (otherwise known as c-Src).

The Src family of non-receptor tyrosine kinases is located intracellularly and is involved in the transmission of biochemical signals such as those that influence tumour cell motility, dissemination and invasiveness and subsequently metastatic tumour growth. Members of the Src family include inter alia c-Src, c-Yes, c-lck and c-Fyn.

It is further known that the Src family of non-receptor tyrosine kinases is highly regulated in normal cells such that, in the absence of extracellular stimuli, the kinases are maintained in an inactive conformation. However, some Src family members, for example c-Src tyrosine kinase, are frequently significantly activated (when compared to normal cell levels) in common human cancers.

Accordingly it has been recognised that an inhibitor of such non-receptor tyrosine kinases should be of value as a selective inhibitor of the motility of tumour cells and as a selective inhibitor of the dissemination and invasiveness of mammalian cancer cells leading to inhibition of metastatic tumour growth. Thus the predominant role of c-Src non-receptor tyrosine kinase is to regulate cell motility which is necessarily required for a localised tumour to progress through the stages of dissemination into the blood stream, invasion of other tissues and initiation of metastatic tumour growth. c-Src kinase is involved in the signal transduction steps which lead to the invasiveness and migratory ability of metastasising tumour cells.

Accordingly Src kinase inhibitors are of value as anti-tumour agents, in particular as selective inhibitors of the motility, dissemination and invasiveness of mammalian cancer cells leading to inhibition of metastatic tumour growth. Particularly, Src kinase inhibitors are of value as anti-invasive agents in the containment and/or treatment of solid tumour disease. Particularly, such compounds are expected to be useful in the prevention or treatment of those tumours which are sensitive to inhibition of one or more of the multiple non-receptor tyrosine kinases such as c-Src kinase that are involved in the signal transduction steps which lead to the invasiveness and migratory ability of metastasising tumour cells. Further, such compounds are expected to be useful in the prevention or treatment of those tumours which are mediated alone or in part by inhibition of the enzyme c-Src, i.e. the compounds may be used to produce a c-Src enzyme inhibitory effect in a warm-blooded animal in need of such treatment. Specifically, such compounds are expected to be useful in the prevention or treatment of solid tumour disease.

Accordingly it has been recognised that an inhibitor of such non-receptor tyrosine kinases should be of value as a selective inhibitor of the motility of tumour cells and as a selective inhibitor of the dissemination and invasiveness of mammalian cancer cells leading to inhibition of metastatic tumour growth. In particular an inhibitor of such non-receptor tyrosine kinases should be of value as an anti-invasive agent for use in the containment and/or treatment of solid tumour disease.

It is known from International Published Patent Application WO 01/94341 that certain 5-position substituted quinazoline derivatives possess Src kinase inhibitory activity and are anti-invasive agents useful in the treatment of various cancers. The compound 4-(6-chloro-2,3-methylenedioxyanilino)-7-[2-(4-methylpiperazin-1-yl)ethoxy]-5-tetrahydropyran-4-yloxyquinazoline is disclosed as Compound No. 73 within Example 14 therein. That compound is a potent Src kinase inhibitor and it is identified herein by way of the code number AZD0530.

The combination of the present invention seeks to provide an improved treatment for the management of cancer by combining a particular Src kinase inhibitor, AZD0530, with a MEK inhibitor.

The present invention also makes use of the particular N3 alkylated benzimidazoles; pyridones; reverse pyridones and pyridazines MEK inhibitors described in International Published Patent Applications WO 03/077914, WO 05/051301 and WO07/044,084.

It is stated in International Published Patent Application WO 03/077914 that the MEK inhibitors disclosed therein may be administered as a sole therapy or may involve, in addition to the compounds of that invention, one or more other anti-tumor substances. However, there is no disclosure of the particular combination of the present invention, nor that any such combination produces surprisingly effective results.

It is also stated in International Published Patent Application WO 05/051301 that the MEK inhibitors disclosed therein may be administered as a sole therapy or may involve, in addition to the compounds of that invention, conventional surgery or radiotherapy or chemotherapy. Such chemotherapy was stated to include one or more of a number of different categories of anti-tumour agents such as other anti-invasion agents (for example metalloproteinase inhibitors like marimastat and inhibitors of urokinase plasminogen activator receptor function). However, there is no disclosure of the particular combination of the present invention, nor that any such combination produces surprisingly effective results.

It is also stated in International Published Patent Application WO 01/94341 that the Src kinase inhibitors disclosed therein may be administered as a sole therapy or may involve, in addition to the quinazoline derivatives of those inventions, conventional surgery or radiotherapy or chemotherapy. Such chemotherapy was stated to include one or more of a number of different categories of anti-tumour agents such as other anti-invasion agents (for example metalloproteinase inhibitors like marimastat and inhibitors of urokinase plasminogen activator receptor function). However, there is no disclosure of the particular combination of the present invention, nor that any such combination produces surprisingly effective results.

In the present invention it has been shown that the inhibition of both Src kinase and MEK kinase results in a reduction of cell invasion. Unexpectedly, it has been found that a particular selection from the generic disclosures of combination therapies mentioned in International Published Patent Applications WO 01/94341, WO 02/16352, WO 03/077914, WO 05/051301 and WO2007/044084 is very effective. In particular, the combination of a MEK inhibitor, or a pharmaceutically-acceptable salt thereof, and the Src kinase inhibitor AZD0530, or a pharmaceutically-acceptable salt thereof, produces surprisingly effective results. More specifically, the combination of a MEK inhibitor and the Src kinase inhibitor AZD0530 produces a greater effect than that achievable by the administration of either a MEK inhibitor or the Src kinase inhibitor AZD0530 alone.

Whilst there is a disclosure in International Published Patent Application WO 05/051301 that the MEK inhibitors disclosed therein may be used in combination with anti-invasive agents there is no specific disclosure of the combination use nor that any such combination produces surprisingly effective results.

Whilst there is a disclosure in International Published Patent Application WO 01/94341 that the Src kinase inhibitors disclosed therein may be used in combination with other anti-invasive agents or anti-proliferative agents, there is no specific disclosure of the combination use of a MEK inhibitor and the Src kinase inhibitor AZD0530, nor that any such combination produces surprisingly effective results.

According to the present invention there is provided a combination suitable for use in the treatment of cancer comprising an MEK inhibitor, or a pharmaceutically acceptable salt thereof, and the Src kinase inhibitor AZD0530, or a pharmaceutically-acceptable salt thereof.

It is to be understood that the term “a combination” envisages the simultaneous, sequential or separate administration of the components of the combination. In one aspect of the invention, “a combination” envisages simultaneous administration of the MEK inhibitor and the Src inhibitor. In a further aspect of the invention, “a combination” envisages sequential administration of those agents. In another aspect of the invention, “a combination” envisages separate administration of those agents. Where the administration of those agents is sequential or separate, the delay in administering the second component should not be such as to lose the benefit of the synergistic effect of the combination therapy. Thus, for the avoidance of doubt, the present invention provides a combination comprising a MEK inhibitor, or a pharmaceutically-acceptable salt thereof, and the Src kinase inhibitor AZD0530, or a pharmaceutically-acceptable salt thereof, for use simultaneously, sequentially or separately in the treatment of cancer, or for use simultaneously, sequentially or separately in the delay of the progression of cancer.

The present invention further provides a combination suitable for use in the treatment of cancer comprising a MEK inhibitor, or a pharmaceutically-acceptable salt thereof, and the Src kinase inhibitor AZD0530, or a pharmaceutically-acceptable salt thereof, for use simultaneously, sequentially or separately for the treatment of cancer.

Suitable MEK inhibitors include those compounds disclosed in International Published Patent Applications WO 99/01426, WO 02/06213, WO 03/077914, WO 05/051301 and WO2007/044084.

Particular MEK inhibitors include the following compounds:—
6-(4-Bromo-2-chloro-phenylamino)-7-fluoro-3-methyl-3H-benzoimidazole-5-carboxylic acid (2,3-dihydroxy-propoxy)-amide;
6-(4-Bromo-2-chloro-phenylamino)-7-fluoro-3-(tetrahydro-pyran-2-ylmethyl)-3H-benzoimidazole-5-carboxylic acid (2-hydroxy-ethoxy)-amide,
1-[6-(4-Bromo-2-chloro-phenylamino)-7-fluoro-3-methyl-3H-benzoimidazol-5-yl]-2-hydroxy-ethanone,
6-(4-Bromo-2-chloro-phenylamino)-7-fluoro-3-methyl-3H-benzoimidazole-5-carboxylic acid (2-hydroxy-1,1-dimethyl-ethoxy)-amide,
6-(4-Bromo-2-chloro-phenylamino)-7-fluoro-3-(tetrahydro-furan-2-ylmethyl)-3H-benzoimidazole-5-carboxylic acid (2-hydroxy-ethoxy)-amide,
6-(4-Bromo-2-fluoro-phenylamino)-7-fluoro-3-methyl-3H-benzoimidazole-5-carboxylic acid (2-hydroxy-ethoxy)-amide and
6-(2,4-Dichloro-phenylamino)-7-fluoro-3-methyl-3H-benzoimidazole-5-carboxylic acid (2-hydroxy-ethoxy)-amide.

More particular MEK inhibitors include:—
6-(4-Bromo-2-chloro-phenylamino)-7-fluoro-3-methyl-3H-benzoimidazole-5-carboxylic acid (2-hydroxy-ethoxy)-amide, referred to hereinafter as MEK inhibitor 1;
2-[(2-fluoro-4-iodophenyl)amino]-N-(2-hydroxyethoxy)-1,5-dimethyl-6-oxo-1,6-dihydropyridine-3-carboxamide; referred to hereinafter as MEK inhibitor 2; and
4-(4-bromo-2-fluorophenylamino)-N-(2-hydroxyethoxy)-1,5-dimethyl-6-oxo-1,6-dihydropyridazine-3-carboxamide or a pharmaceutically acceptable salt thereof.

Thus, in a further aspect the present invention provides a combination suitable for use in the treatment of cancer comprising MEK inhibitor 1, or a pharmaceutically acceptable salt thereof, or MEK inhibitor 2, or a pharmaceutically acceptable salt and the Src kinase inhibitor AZD0530, or a pharmaceutically acceptable salt thereof.

A suitable pharmaceutically-acceptable salt of the MEK inhibitor or AZD0530 is, for example, a pharmaceutically-acceptable acid-addition salt, for example an acid-addition salt with an inorganic or organic acid such as hydrochloric, hydrobromic, sulphuric, trifluoroacetic, citric, maleic or fumaric acid, for example a mono- or di-fumaric acid salt.

When the MEK inhibitor is MEK inhibitor 1, a preferred salt is the hydrogen sulphate salt. The hydrogen sulphate salt of MEK inhibitor 1 may be synthesised according to the processes described in WO07/076,245.

The cancer treatment of the present invention includes an anti-tumour effect that may be assessed by conventional means such as the response rate, the time to disease progression and/or the survival rate. Anti-tumour effects of the present invention include, but are not limited to, inhibition of tumour growth, tumour growth delay, regression of tumour, shrinkage of tumour, increased time to regrowth of tumour on cessation of treatment and delay of disease progression. For example, it is expected that when the combination of the present invention is administered to a warm-blooded animal such as a human, in need of treatment for cancer involving a solid tumour, the treatment will produce a beneficial effect, as measured by, for example, one or more of the extent of the anti-tumour effect, the response rate, the time to disease progression and the survival rate. As previously stated, it is believed that the combination of the present invention will provide a beneficial or synergistic effect on the treatment or prophylaxis of cancer or it will provide a “synergistic treatment” of cancer. According to the present invention, a combination treatment is defined as affording a “synergistic effect” or a “synergistic treatment” if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with the MEK inhibitor alone or the Src kinase inhibitor AZD0530. Further, the effect of the combination is synergistic if a beneficial effect is obtained in a group of patients that does not respond (or responds poorly) to the MEK inhibitor or Src kinase inhibitor AZD05030 alone. In addition, the effect of the combination treatment is defined as affording a synergistic effect if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment. In particular, synergy is deemed to be present if the conventional dose of the MEK inhibitor or the Src kinase inhibitor AZD0530 may be reduced without detriment to one or more of the extent of the response, the response rate, the time to disease progression and survival data, in particular without detriment to the duration of the response, but with fewer and/or less troublesome side-effects than those that occur when conventional doses of each component are used.

Thus, in a further aspect, the present invention provides a combination for use in the synergistic treatment of cancer comprising a MEK inhibitor, or a pharmaceutically-acceptable salt thereof, and the Src kinase inhibitor AZD0530, or a pharmaceutically-acceptable salt thereof. In a further aspect the present invention provides a combination for use in the synergistic treatment of cancer comprising MEK inhibitor 1, or a pharmaceutically acceptable salt thereof, or MEK inhibitor 2, or a pharmaceutically acceptable salt, or a pharmaceutically-acceptable salt thereof, and the Src kinase inhibitor AZD0530, or a pharmaceutically-acceptable salt thereof.

The therapeutic combination of the present invention may be administered in the form of a suitable pharmaceutical composition. According to this aspect of the invention there is provided a pharmaceutical composition suitable for use in the treatment of cancer which comprises a combination as defined hereinbefore in association with a pharmaceutically-acceptable excipient or carrier.

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention, the MEK inhibitor and the Src kinase inhibitor AZD0530 of the combination treatment may be delivered endoscopically, intratracheally, intralesionally, percutaneously, intravenously, subcutaneously, intraperitoneally or intratumourally. Preferably, the MEK inhibitor is administered orally. Preferably the Src kinase inhibitor AZD0530 is administered orally. In general the compositions described herein may be prepared in a conventional manner using conventional excipients. The compositions of the present invention are advantageously presented in unit dosage form.

The MEK inhibitor will generally be administered so that a daily dose in the range of, for example, 0.1 mg/kg to 75 mg/kg body weight is received, given if required in divided doses.

The Src kinase inhibitor AZD0530 will generally be administered so that a daily dose in the range of, for example, 0.1 mg/kg to 75 mg/kg body weight is received, given if required in divided doses. In general, lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

The dosages and schedules described hereinbefore may be varied according to the particular disease state and the overall condition of the patient. For example, it may be necessary or desirable to reduce the above-mentioned doses of the components of the combination treatment in order to reduce toxicity. Dose scheduling can be determined by the practitioner who is treating any particular patient using his professional skill and knowledge.

It will be appreciated that the pharmaceutical composition according to the present invention includes a composition comprising a MEK inhibitor and the Src kinase inhibitor AZD0530 and a pharmaceutically-acceptable excipient or carrier. Such a composition conveniently provides the therapeutic combination product of the invention for simultaneous administration in the treatment of cancer.

According to this aspect of the invention there is provided a pharmaceutical composition suitable for use in the treatment of cancer which comprises a MEK inhibitor or a pharmaceutically acceptable salt thereof, the Src kinase inhibitor AZD0530, or a pharmaceutically-acceptable salt thereof, and a pharmaceutically-acceptable excipient or carrier.

A pharmaceutical composition according to the present invention also includes separate compositions comprising a first composition comprising a MEK inhibitor, or a pharmaceutically-acceptable salt thereof, and a pharmaceutically-acceptable excipient or carrier, and a second composition comprising the Src kinase inhibitor AZD0530, or a pharmaceutically-acceptable salt thereof, and a pharmaceutically-acceptable excipient or carrier. Such a composition conveniently provides the therapeutic combination of the invention for sequential or separate administration in the treatment of cancer but the separate compositions may also be administered simultaneously.

Conveniently such a pharmaceutical composition of the invention comprises a kit comprising a first container with a suitable composition containing the MEK inhibitor and a second container with a suitable composition containing the Src kinase inhibitor AZD0530. According to this aspect of the present invention there is provided a kit for use in the treatment of cancer comprising:—
a) a MEK inhibitor, or a pharmaceutically acceptable salt thereof, together with a pharmaceutically-acceptable excipient or carrier, in a first unit dosage form (such as a tablet or capsule);
b) the Src kinase inhibitor AZD0530, or a pharmaceutically-acceptable salt thereof, together with a pharmaceutically-acceptable excipient or carrier, in a second unit dosage form; and
c) container means for containing said first and second unit dosage forms.

According to a further aspect of the present invention there is provided the use of a combination as defined hereinbefore in the manufacture of a medicament for administration to a warm-blooded animal to provide the treatment of cancer.

According to a further aspect of the present invention there is provided a combination suitable for use in the synergistic treatment of cancer comprising an MEK inhibitor, or a pharmaceutically acceptable salt thereof, and the Src kinase inhibitor AZD0530, or a pharmaceutically-acceptable salt thereof.

According to a further aspect of the present invention there is provided a method for the treatment of cancer which comprises the administration to a warm-blooded animal that is in need of such treatment of effective amounts of the components of the combination as defined hereinbefore.

According to this aspect of the present invention there is also provided a method for the cancer which comprises the administration to a warm-blooded animal that is in need of such treatment of an effective amount of a MEK inhibitor, or a pharmaceutically-acceptable salt thereof, as defined hereinbefore, before simultaneously with or after the administration of an effective amount of the Src kinase inhibitor AZD0530, or a pharmaceutically-acceptable salt thereof.

According to this aspect of the present invention there is also provided a method for the treatment of cancer which comprises the simultaneous, sequential or separate administration to a warm-blooded animal that is in need of such treatment of effective amounts of the components of the combination as defined hereinbefore.

According to this aspect of the present invention there is also provided a method for the treatment of cancer which comprises the administration to a warm-blooded animal that is in need of such treatment of an effective amount of a MEK inhibitor, or a pharmaceutically-acceptable salt thereof, as defined hereinbefore and the simultaneous, sequential or separate administration of an effective amount of the Src kinase inhibitor AZD0530, or a pharmaceutically-acceptable salt thereof.

According to a further aspect of the invention there is provided a method for the treatment of malignant or metastatic melanoma in a warm-blooded animal, such as a human, which comprises administering to said animal an effective amount of a MEK inhibitor or a pharmaceutically-acceptable salt thereof, as defined hereinbefore, before simultaneously with or after the administration of an effective amount of the Src kinase inhibitor AZD0530, or a pharmaceutically-acceptable salt thereof.

According to a further aspect of the invention there is provided a method for the treatment of non-small cell lung cancer (NSCLC) in a warm-blooded animal, such as a human, which comprises administering to said animal an effective amount of a MEK inhibitor or a pharmaceutically-acceptable salt thereof, as defined hereinbefore, before, simultaneously with or after the administration of an effective amount of the Src kinase inhibitor AZD0530, or a pharmaceutically-acceptable salt thereof.

According to a further aspect of the present invention there is provided a method for the treatment of a cancer in a warm-blooded animal, such as a human, which comprises administering to said animal an effective amount of a MEK inhibitor or a pharmaceutically acceptable salt thereof, before, after or simultaneously with an effective amount of the Src kinase inhibitor AZD0530, or a pharmaceutically acceptable salt thereof; wherein the MEK inhibitor and AZD0530 may each optionally be administered together with a pharmaceutically acceptable excipient or carrier.

According to a further aspect of the present invention there is provided a combination comprising a MEK inhibitor, or a pharmaceutically acceptable salt thereof, and the Src kinase inhibitor AZD0530, or a pharmaceutically-acceptable salt thereof, for use in treating cancer.

Combination treatments of the present invention are expected to be particularly useful in the prophylaxis and treatment of diseases such as cancer and Kaposi's sarcoma. In particular such combination treatments of the invention are expected to be useful in the treatment of cancer, for example cancer of the lung, head and neck, brain, colon, rectum, oesophagus, stomach, liver, biliary tract, thyroid, kidney, cervix, ovary, uterus, skin, breast, bladder, prostate, pancreas and including haematological malignancies such as leukaemia, multiple myeloma and lymphoma. In particular such combination treatments of the invention are expected to slow advantageously the growth of primary and recurrent solid tumours of, for example, the colon, rectum, pancreas, brain, bladder, ovary, breast, prostate, lungs, liver and skin. Combination treatments of the present invention are expected to slow advantageously the growth of tumours in malignant or metastatic melanoma, colorectal cancer, pancreatic cancer, hepatocellular cancer and lung cancer including non-small cell lung cancer (NSCLC). Combination treatments of the present invention are expected to slow advantageously the growth of tumours in malignant or metastatic melanoma, colorectal cancer, pancreatic cancer and lung cancer including non-small cell lung cancer (NSCLC). More especially combination treatments of the present invention are expected to slow advantageously the growth of tumours in malignant or metastatic melanoma. More especially combination treatments of the present invention are expected to slow advantageously the growth of tumours in non-small cell lung cancer (NSCLC). More especially combination treatments of the present invention are expected to slow advantageously the growth of tumours in hepatocellular cancer.

A combination treatment of the present invention as defined hereinbefore may be administered as a sole therapy or may in addition involve surgery or radiotherapy or the administration of a chemotherapeutic agent. Other chemotherapeutic agents for optional use with a combination treatment of the present invention include those described in WO 07/076,245, which is incorporated herein by reference. Such chemotherapy may cover an agent from one of the following categories:—

(i) antiangiogenic agents

(ii) vascular targeting agents

(iii) cytostatic agents

(iv) other anti-invasion agents

(v) inhibitors of growth factor function

(vi) antiproliferative/antineoplastic drugs

(vii) biological response modifiers

(viii) antibodies

(ix) antisense therapies

(x) gene therapy approaches and

(xi) immunotherapy approaches.

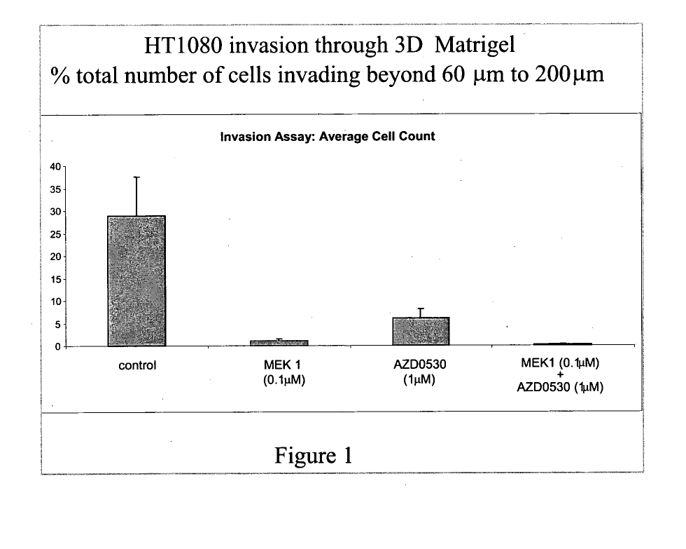

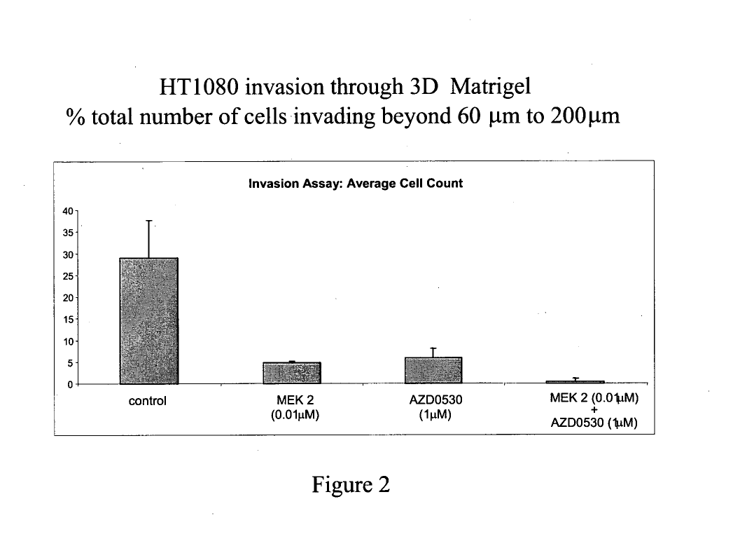

FIGS. 1 and 2 shows the % HT1080 cells invading between 60-200 μm depth of 3D Matrigel

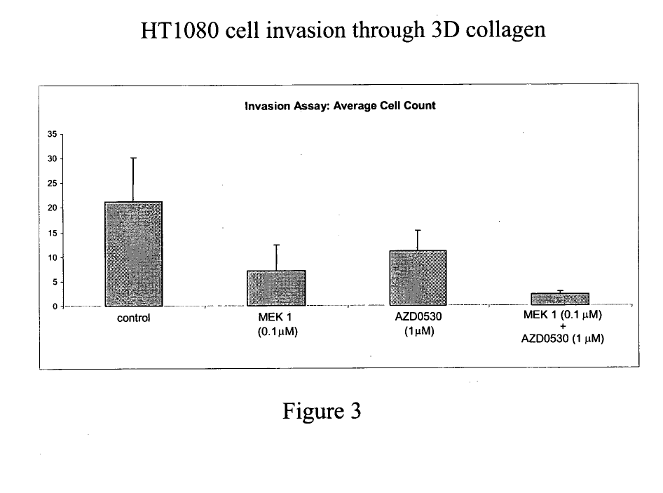

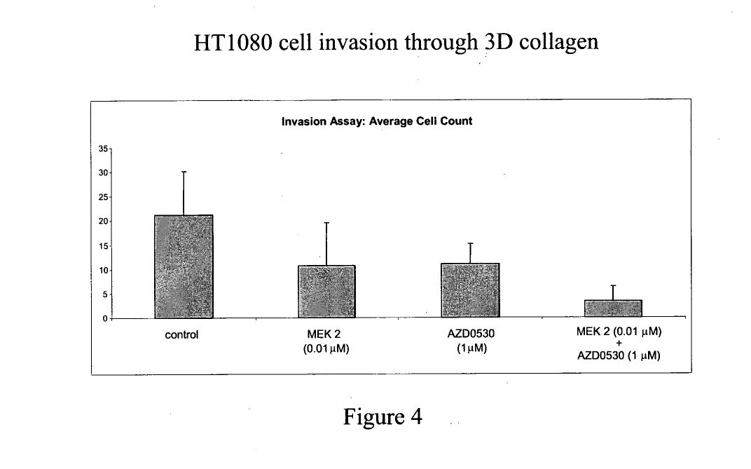

FIGS. 3 and 4 shows the % HT1080 cells invading between 60-200 μm depth of 3D Collagen.

MEK inhibitor 1 (referred to as MEK 1 on the figures) is 6-(4-bromo-2-chloro-phenylamino)-7-fluoro-3-methyl-3H-benzoimidazole-5-carboxylic acid (2-hydroxy-ethoxy)-amide. The compound is described in International Patent Publication Number WO03/077914, within example 10.

MEK inhibitor 2 (referred to as MEK 2 on the figures) is 2-[(2-fluoro-4-iodophenyl)amino]-N-(2-hydroxyethoxy)-1,5-dimethyl-6-oxo-1,6-dihydropyridine-3-carboxamide. This compound is prepared by the following method:—

Step a: Preparation of methyl 2-(2-fluoro-4-iodophenylamino)-1,5-dimethyl-6-oxo-1,6-dihydropyridine-3-carboxylate: To a solution of 2-fluoro-4-iodobenzeneamine (0.058 g, 0.31 mmol) in THF (2 mL) at −78° C. under N₂ was added lithium bis(trimethylsilyl)amide (0.56 mL, 0.56 mmol, 1M solution in hexanes) dropwise. The reaction mixture was stirred for one hour at −78° C. Methyl 2-chloro-1,5-dimethyl-6-oxo-1,6-dihydropyridine-3-carboxylate (0.060 g, 0.28 mmol) was then added dropwise as a solution in THF (1 mL) and the reaction mixture was stirred for 25 minutes at −78° C. The reaction mixture was quenched by the addition of H₂O and the pH was adjusted with 0.1M HCl and then diluted with EtOAc and saturated NaCl and the layers separated. The aqueous layer was back extracted with EtOAc (1×). The combined EtOAc layers were dried (Na₂SO₄) and concentrated under reduced pressure. Purification by flash column chromatography (methylene chloride/EtOAc, 20:1) gave 0.086 g (84%) pure desired product as a white crystalline solid. MS ESI (+) m/z 417 (M+1) detected; ¹H NMR (400 MHz, CDCl₃) δ 9.56 (s, 1H), 7.79 (s, 1H), 7.49 (d, 11H), 7.36 (d, 1H), 6.43 (t, 1H), 3.85 (s, 3H), 3.30 (s, 3H), 2.15 (s, 3H).
Step b: Preparation of 2-(2-fluoro-4-iodophenylamino)-1,5-dimethyl-6-oxo-N-(2-(vinyloxy)ethoxy)-1,6-dihydropyridine-3-carboxamide: To a solution of methyl 2-(2-fluoro-4-iodophenylamino)-1,5-dimethyl-6-oxo-1,6-dihydropyridine-3-carboxylate (0.500 g, 1.20 mmol) in THF (60 mL) was added O-(2-vinyloxy-ethyl)-hydroxylamine (0.149 g, 1.44 mmol). The solution was cooled to 0° C. and lithium bis(trimethylsilyl)amide (4.81 ml, 4.81 mmol) (1M solution in hexanes) was added dropwise. The reaction mixture was warmed to room temperature. After stirring for 10 minutes the reaction mixture was quenched by the addition of 1M HCl and partitioned between EtOAc and saturated NaCl. The layers were separated and the organic layer was dried (Na₂SO₄) and concentrated under reduced pressure to yield a crude yellow solid that was used without purification in the next step.
Step c: Preparation of 2-(2-fluoro-4-iodophenylamino)-N-(2-hydroxyethoxy)-1,5-dimethyl-6-oxo-1,6-dihydropyridine-3-carboxamide: To a solution of crude 2-(2-fluoro-4-iodophenylamino)-1,5-dimethyl-6-oxo-N-(2-(vinyloxy)ethoxy)-1,6-dihydropyridine-3-carboxamide (0.585 g, 1.20 mmol) in ethanol (10 mL) was added aqueous 2 M HCl (3 mL). The reaction mixture was stirred for 45 minutes at room temperature. The pH of the reaction mixture was adjusted to pH 7 with 1M NaOH. The reaction mixture was diluted with EtOAc and H₂O. The organic layer was separated and washed with saturated NaCl. The combined aqueous layers were back extracted with EtOAc (1×). The combined organic layers were dried (Na₂SO₄) and concentrated under reduced pressure. Purification by silica gel flash column chromatography (methylene chloride/MeOH, 15:1) gave 2-(2-fluoro-4-iodophenylamino)-N-(2-hydroxyethoxyl)-1,5-dimethyl-6-oxo-1,6-dihydropyridine-3-carboxamide (0.421 g; 76% over two steps) as a pale yellow solid. MS ESI (+) m/z 462 (M+1) pattern detected; ¹H NMR (400 MHz, CDCl₃) δ 9.77 (s, 1H), 8.50 (s, 1H), 7.47 (d, 1H), 7.36 (d, 1H), 6.43 (t, 1H), 4.04 (br s, 2H), 3.85 (br s, 1H), 3.74 (br s, 2H), 3.29 (s, 3H), 2.14 (s, 3H).

### EXAMPLES

### Biological Test Procedures

The following test methods may be used to demonstrate the activity of a MEK inhibitor when used in combination with the Src kinase inhibitor AZD0530.

### Cell Invasion

The ability of the Src kinase inhibitor AZD0530, MEK inhibitor 1, MEK inhibitor 2, and a combination of the Src kinase inhibitor AZD0530 and either MEK inhibitor 1 or MEK inhibitor 2 to inhibit invasion of HT1080 cells through both 3D Matrigel and 3D collagen substrate was measured by the following method.

HT1080 cells are a human fibrosarcoma cell line isolated in 1971 from a 35 year old Male caucasian. Our HT1080 cells where supplied from the Tissue Culture Unit at Alderley Park. The cells are routinely cultured in DMEM (Dulbecco's Modified Eagle's Medium) supplemented with 10% FCS (Fetal Calf Serum) and 2 mM L-Glutamine. Matrix substrates used for invasion assays composed of either fibrillar collagen or Matrigel™ basement membrane were prepared as follows: —

### i) Fibrillar Collagen

A collagen solution was prepared by gently mixing the following components at room temperature

| | |
|---|---|
| Vitrogen 100 (collagen type I) | 2 ml |
| 10 x Dulbecco's Modified Eagle's Medium(DMEM) | 0.525 ml |
| Sterile water | 2.9 ml |
| 0.1N NaOH | 0.5 ml |

The collagen solution was gently mixed and 80 μl was added to the upper chamber of a transwell insert. The transwell inserts (Corning Incorporated) were a “24-well” plate format. Each insert consisted of a polycarbonate membrane with 8 μm pore size and a membrane diameter is 6.5 mm. This was then allowed to set at 37° C. for 24 hours in a sterile environment.

### ii) Matrigel™

The Matrigel was defrosted on ice in a fridge overnight. 80 μl was added to the upper chamber of a transwell insert (24 well plate format). This was then incubated at 37° C. for 90 min in a sterile environment.

### Cell Attachment Assay

The HT1080 cells were re-suspended using cell-dissociation solution (Sigma) at 1×10⁵ cell/ml in DMEM (0.2% foetal calf serum (FCS)+L-Glutamine) media. Compound doses (0.1 μM MEK1, 0.01 μM MEK2 and 1 μM AZD0530 as appropriate) were added to 1 ml aliquots of cell suspension. Dimethylsulphoxide (DMSO) (0.1%) was added to 1 ml aliquots of cell suspension to provide the control.

750 μl of DMEM (10% FCS+L-Glutamine) supplemented with compound doses (0.01, 0.1 and 1 mM) or 0.1% DMSO (control) were added to the lower chamber of the transwell insert. Aliquot 100 μl (1×10⁴ cells) of control and compound treated cell suspension was added to the upper chamber of the Matrigel and Collagen containing transwell inserts. The transwell inserts were then incubated at 37° C. for 72 hours. The cell nuclei were then labelled by immersing the transwell inserts into 24 well plates containing 0.5 ml DMEM (10% FCS+L-Glutamine)+101M Hoechst 33342 (supplied by Invitrogen. 0.5 ml DMEM (10% FCS+L-Glutamine)+101M Hoechst was then added to the upper chamber. The plates containing the transwell inserts were then incubated for 30 minutes at 37° C. under sterile conditions. The transwells were then fixed by immersing the plates in −20° C. methanol for 10 minutes at room temperature. The transwells were then tipped and blotted and washed twice in phosphate-buffered serum (PBS). They were then stored in PBS at 4° C. what until analysed by a confocal microscope system.

### Confocal and Image Analysis

Using a 20× objective on a Bio-Rad Radiance 2000 multiphoton confocal microscope, optical images of cells on top of collagen (or Matrigel) gels and invaded cells at sequential 20 μm sections through the collagen (or Matrigel) gel where obtained. Images where imported into Image-Pro-Plus image analysis software to calculate pixels at each optical section to obtain a relative value of cell number at each section. Data was exported to Microsoft Excel and expressed as the percentage total number of cells calculated in all optical sections per sample that has invaded beyond a specified distance (e.g. 60-200 μm) or at a specified distance (e.g. 60 μm). Mean values from triplicate samples + and − standard deviation where calculated. When the Src kinase inhibitor AZD0530 was used in combination with either MEK inhibitor 1 or 2 increased activity was demonstrated over that seen with either, MEK inhibitor 1 or 2 or AZD0530 alone.

1. A combination suitable for use in the treatment of cancer comprising a MEK inhibitor, or a pharmaceutically acceptable salt thereof, and 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable salt thereof.

2. A combination for use in the synergistic treatment of cancer comprising a MEK inhibitor, or a pharmaceutically acceptable salt thereof, and 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable salt thereof.

3. A pharmaceutical composition suitable for use in the treatment of cancer which comprises a combination according to claim 1 in association with a pharmaceutically-acceptable excipient or carrier.

4. (canceled)

5. A pharmaceutical composition according to claim 3 which comprises a first composition comprising a MEK inhibitor, or a pharmaceutically acceptable salt thereof, and a pharmaceutically acceptable carrier, and a second composition comprising 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable salt thereof, and a pharmaceutically-acceptable excipient or carrier.

6. (canceled)

7. (canceled)

8. (canceled)

9. A method for the treatment of cancer in a patient in need of such treatment, the method comprising administering to the patient a combination of a therapeutically effective amount of a MEK inhibitor, or a pharmaceutically acceptable salt thereof, and a therapeutically effective amount of 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable salt thereof.

10. A combination according to claim 1 wherein the MEK inhibitor is selected from 6-(4-Bromo-2-chloro-phenylamino)-7-fluoro-3-methyl-3H-benzoimidazole-5-carboxylic acid (2-hydroxy-ethoxy)-amide or a pharmaceutically-acceptable salt thereof, or 2-[(2-fluoro-4-iodophenyl)amino]-N-(2-hydroxyethoxy)-1,5-dimethyl-6-oxo-1,6-dihydropyridine-3-carboxamide or a pharmaceutically-acceptable salt thereof.

11. A pharmaceutical composition according to claim 3 wherein the MEK inhibitor is selected from 6-(4-Bromo-2-chloro-phenylamino)-7-fluoro-3-methyl-3H-benzoimidazole-5-carboxylic acid (2-hydroxy-ethoxy)-amide or a pharmaceutically-acceptable salt thereof or 2-[(2-fluoro-4-iodophenyl)amino]-N-(2-hydroxyethoxy)-1,5-dimethyl-6-oxo-1,6-dihydropyridine-3-carboxamide or a pharmaceutically-acceptable salt thereof.
